(19) United States
(12) Patent Application Publication
(10) Pub. No.: US 2007/0282226 A1
(43) Pub. Date: Dec. 6, 2007

(54) Apparatuses, Systems and Methods for Monitoring Conditions in an Oral Cavity

(76) Inventor: William H. Longley, Atlanta, GA (US)

Correspondence Address: Robert A. Blaha;SMITH FROHWEIN TEMPEL, GREENLEE BLAHA LLC, P.O. Box 88148, Atlanta, GA, 30346, US

(21) Appl. No.: 11/531,620

(22) Filed: Sep. 13, 2006

Related U.S. Application Data
(63) Continuation-in-part of application No. 11/422,279, filed on Jun. 5, 2006.

(51) Int. Cl. A61B 5/103

(52) U.S. Cl. 600/587

(57) ABSTRACT

An apparatus constructed with a housing that encloses an array of sensors, a memory and a transceiver is attached in an oral cavity. The apparatus responds to commands received via the transceiver. When so directed, the apparatus uses one or more sensors alone or in combination to record measurements responsive to the presence of a select substance or substances in a host's oral cavity. When communicatively coupled to a suitably configured communication device, the apparatus receives commands that direct the apparatus to transmit the recorded data, or both an identifier and the data. The transmitted data can be used to determine whether the user of the apparatus has complied with a recommended consumption behavior, has good oral hygiene, is suffering from a disease, etc.

### CROSS REFERENCE To RELATED APPLICATION

This application is a continuation-in-part of U.S. patent application Ser. No. 11/422,279, entitled “Apparatuses, Systems and Methods for Confirming Use of an Apparatus,” filed on Jun. 5, 2006, the entire disclosure of which is incorporated herein by reference.

### BACKGROUND

The human mouth is the gateway to the body. A host of different items can be consumed via the mouth. For example, people eat, drink, and take prescribed medications via the mouth. While food, water, and prescribed medications generally promote the development and maintenance of a healthy body, other consumed substances such as smoke, drugs, alcohol, etc. can interfere with human health and can lead to powerful unhealthy addictions.

In addition to smoke and drugs, some people develop unhealthy eating habits, such as the over or under consumption of food in general or the over or under consumption of one or more of the basic dietary constituents. Our diets include a mix of proteins, lipids (fats) and carbohydrates (sugars). These basic dietary components should be consumed in an appropriate balance to maintain healthy cell growth and function.

A number of therapies have been developed related to the treatment of eating disorders, drug use (including the use of alcohol and smoking cessation programs) and other medical issues.

To be effective, a prescribed or self-motivated therapy should be followed until a desired benefit or result is achieved. A number of therapies rely on patient self-control, self-monitoring and recording to verify compliance with the therapy.

For example, one method of smoking cessation uses a transdermal patch that delivers nicotine to the bloodstream through a mechanism other than inhaling the nicotine-bearing smoke, which contains numerous other chemicals and toxins. Other methods use nicotine laced gum, inhalers or sublingual tablets. These alternative nicotine delivery mechanisms rely on the patient's self-control to not smoke and in many cases ingest or inhale the substitute in accordance with a desired schedule and dose.

Despite the availability of these smoking cessation aids, the success rate among motivated smokers to quit smoking is still undesirably low. Several authors have suggested that behavioral modification techniques may be helpful. One behavioral modification method is a self-administered gradual reduction of smoking. Several methods and/or devices have been used to achieve a gradual reduction in smoking. In the early 1970s, timed prompts and audible cues to alert a smoker as to the next permitted time to smoke were employed. (Shapiro et al. 1971. “Smoking on cue. A behavioral approach to smoking reduction,” J. Health Social Behavior 12: 108-113; Bernard, H. S. and Efron, J. A. 1972. “Eliminating versus reducing smoking using pocket timers,” Behavior Research Therapy 10:39-41). While some studies reported use of such self-administered controlled smoking devices resulted in significant decreases in average cigarette consumption, there were significant problems associated with their use. Reduction methods are commonly compromised when smokers control the timing of their cigarettes and fail to exercise coping skills when confronted with usual smoking cues. Methods to verify compliance of such programs typically rely on the smoker to register each smoking event.

Similar problems exist with verification of a patient's compliance with a prescribed diet. For example, many diets include a meal plan or schedule of foods to be consumed by the dieter. Despite the availability of these diets, the success rate among motivated dieters to achieve desired weight management goals is still undesirably low. Diets, like smoking cessation plans are commonly compromised when dieters consume foods not included in the diet or consume permitted foods in quantities that exceed a recommended amount. Again, methods to verify compliance with most diets rely on the dieter to keep a record of consumed items.

Problems also exist with verification of a patient's compliance with a prescribed medication. For example, many medications are provided by a pharmacy in a liquid suspension in a container labeled with the prescribing physician's instructions regarding dose and regularity. Again, methods to verify a patient's compliance with the prescribed usage schedule rely on the patient or the patient's care giver to adhere to the schedule. Further problems exist with acquiring any information at all regarding an individual's consumption of non-prescription drugs and alcohol.

Furthermore, an operator of machinery, including transportation systems, that does not show signs of addiction to alcohol or other drugs might have uncharacteristically consumed or smoked one or more drugs that may adversely affect their ability to perform required tasks safely.

Regardless of the therapy or the consumed item, present compliance verification methods are problematic because they rely on costly, invasive or inconvenient procedures such as those that require the collection, handling and testing of bodily fluids or they rely on the patient to keep a timely and accurate record of their own behavior. Testing methods are untimely as tests are performed after the consumption event has occurred. A patient or host generated record is inconvenient and is subject to errors of omission and memory when entries are made after the consumption event has occurred. Accordingly, patients, professionals, insurers, parents and others interested in an individual's consumption behaviors over time recognize the paramount importance of developing solutions for monitoring and identifying various consumption behaviors in a manner that avoids the shortcomings of present techniques.

### SUMMARY

An embodiment of an apparatus that senses, records and transfers information concerning the environment in an oral cavity comprises a housing with a power source, a sensor assembly, a memory and a transceiver. The housing is configured for attachment within an oral cavity. The power source supplies power to as required by sensor assembly, memory and transceiver. The sensor assembly is sensitive to attributes of a select substance or substances in the oral cavity. The memory is coupled to the sensor assembly and stores information responsive to the presence or concentration of a substance in the oral cavity. The transceiver is coupled to the memory and configured to communicate the information in accordance with a received command.

An embodiment of a system for monitoring conditions and the presence of substances in an oral cavity comprises an apparatus, communication device and a collector. The apparatus is attached within the oral cavity such that the apparatus cannot be removed by the host. The apparatus is configured to identify and store information responsive to the presence of a substance or substances in the oral cavity. The apparatus is configured to record information at random or predetermined intervals. The communication device is configured to establish a communication session with the apparatus and direct the apparatus to communicate the recorded information. The collector, which can be embodied in a multitude of computing device form factors, receives and stores the information from the communication device.

An embodiment of a method for monitoring conditions in an oral cavity comprises the steps of providing an apparatus suitable for being fixed within an oral cavity, using the apparatus to identify and record information responsive to the presence of a select substance or substances in the oral cavity, and using a communication device to establish a communication session with the apparatus to retrieve the recorded information.

Other apparatuses, systems, methods, features and advantages will be or will become apparent to one with skill in the art upon examination of the following figures and detailed description. All such additional apparatuses, systems, methods, features and advantages are defined and protected by the accompanying claims.

### BRIEF DESCRIPTION OF THE FIGURES

The apparatuses, systems and methods for monitoring an oral cavity can be better understood with reference to the following figures. The components within the figures are not necessarily to scale; emphasis instead is placed upon clearly illustrating the principles that support the apparatuses, systems and methods. Moreover, in the figures, like reference numbers designate corresponding parts throughout the different views.

FIG. 1 is a schematic diagram illustrating components of a system for monitoring conditions in an oral cavity.

FIGS. 2A-2D are perspective views of example embodiments of the apparatus of FIG. 1.

FIG. 3 is a schematic diagram illustrating an embodiment of the communication device of FIG. 1.

FIG. 4 is a schematic diagram illustrating an embodiment of the apparatus of FIG. 1

FIG. 5 is a schematic diagram illustrating an embodiment of the sensor assembly of FIG. 4.

FIG. 6 is a schematic diagram illustrating an alternative embodiment of the sensor assembly of FIG. 4.

FIG. 7 is a flow diagram illustrating an embodiment of a method for monitoring conditions in an oral cavity.

FIG. 8 is a flow diagram illustrating an alternative embodiment of a method for monitoring conditions in an oral cavity.

### DETAILED DESCRIPTION

An apparatus, system and method provide not only a passive means of recording conditions indicative of the presence of one or more select substances in an oral cavity but also a means by which compliance with therapies associated with diminution in smoking or oral consumption of select substances including dietary constituents and drugs can be independently confirmed. In some embodiments, the apparatus, system and method indirectly identify and record the presence of a substance by detecting a byproduct in the oral cavity (e.g., an intoxilyzer). In other embodiments, the apparatus directly detects and records the presence of a target substance (e.g., a micro-electromechanical system (MEMS) sensor configured to detect a concentration of salt). The apparatus is configured to accumulate data until it receives a command to transfer the recorded information.

In preferred embodiments, the apparatus records an indication of the time and whether one or more select substances are present in the oral cavity in response to an internally generated signal. The apparatus is self-contained and fixed in the host's oral cavity. In some embodiments, the apparatus is installed by a healthcare professional using approved mounting hardware or adhesives suitable for installing dental fixtures in a host's oral cavity. In these embodiments, the apparatus cannot be easily removed by the host. In other embodiments, the apparatus is configured to be self-installed or installed by a parent or caregiver. In these other embodiments, the apparatus can be configured with mechanisms that make interference with, removal or other tampering with the device evident. Regardless of the attachment method or mechanism, the apparatus is devoid of host serviceable controls. Consequently, the apparatus provides for greater accuracy and completeness of records concerning the host's oral consumption. The host need only agree to have the apparatus placed in the user's oral cavity.

When in the presence of a suitably configured communication device, the apparatus responds to a command to provide collected information regarding the presence of a select substance in the oral cavity. The communication device is coupled to a data collector (e.g., a computer) with a data-storage device. The collected and stored information can be accessed from the data-storage device for analysis by those with access privileges. In addition, collected information from subsequent communication sessions can be appended to earlier stored data from an identified host of the apparatus.

Some hosts outfitted with the apparatus will immediately adjust their oral consumption habits to conform to a prescribed therapy or diet. It is believed that some other hosts will adjust their oral consumption habits once they receive accurate information regarding their consumption habits and know that they cannot cheat.

The apparatus and method can also be used for the diagnosis of eating disorders, to determine if the host is under the influence of alcohol, or other habit-forming or debilitating substances.

The apparatus is constructed with a housing or chamber that substantially encloses a power source, sensor assembly, a memory, and a transceiver. The apparatus responds to commands received via the transceiver. The apparatus is reset or otherwise configured to record a measurement or measurements. Measurements can be recorded in accordance with a predetermined interval throughout the entire data collection period, in accordance with different intervals depending upon actual conditions in the oral cavity or the time of day, or random intervals. When accumulated data exceeds the storage capacity of the internal memory, the apparatus overwrites the oldest recorded data.

The apparatus is prescribed or otherwise provided and installed in a host's oral cavity. Preferably, the apparatus is installed via mounting hardware or adhesives, such that the apparatus cannot be removed by the host. Before the capacity of the installed memory or power supply in the apparatus has been exhausted, the host returns to the provider or another designated party with a suitably configured communication device. The communication device is used to establish a wireless communication session with the apparatus. A command received from the communication device directs the apparatus to transfer the recorded information to the communication device. The communication device may include a memory of like or greater capacity than the memory in the apparatus. Thus, the communication device can temporarily store the recorded data.

Alternatively, the communication device is coupled to a suitably configured collector such as a desktop computer or other computing device. When coupled to a collector, recorded data from the apparatus can be received and transferred to the collector in a single communication session. Output devices coupled to the collector can be used by those with access privileges to observe what substances were present in the host's oral cavity and when they were present. The transmitted measurements can be used to determine whether the host was compliant with a recommended therapy, diet, under the influence of alcohol, consuming illicit drugs, suffering from one or more eating disorders, or suffering from other medical conditions.

The sensor assembly is configured such that it is sensitive to one or more conditions in the host's oral cavity. For example, the sensor assembly may be sensitive to a range of temperatures. Generally, the apparatus will have the same temperature as the oral cavity. In most hosts, the oral cavity is approximately ninety-eight to ninety-nine degrees Fahrenheit. By periodically recording the temperature of the apparatus, it can be determined with a fairly high degree of certainty when the host was consuming hot or cold food or beverages. By way of further example, the sensor assembly may be sensitive to other conditions that can be expected in an oral cavity such as hydrogen ion concentration or pH; the presence of gaseous compounds including smoke, ethanol, inhalants, etc.; the presence of solid and liquid compounds including alcohol, other drugs, salt, proteins, lipids, and carbohydrates.

A temperature sensor can be entirely contained within a housing or cavity formed by a dental acrylic or other material approved for use in an oral cavity. For example, a TS20 temperature sensor is a high-precision complementary metal-oxide semiconductor (CMOS) temperature sensor that provides for high-accuracy low-power temperature monitoring. With a supply voltage of 2.4V to 6V, the aTS20 is accurate to ±3° C. over a temperature range of −40° C. to 125° C. and has a typical room temperature accuracy of ±0.5° C. Reducing the supply voltage to 2.4V does not change the negative and positive temperature extremes. In addition, the TS20 does not require external calibration. Calibration of each device is performed at the factory. The TS20 is available from Andigilog of Tempe, Ariz., U.S.A.

The logarithmic pH scale is a measure of the number of moles of hydrogen ions (H⁺) per liter of solution or molarity. A pH sensor comprises measurement and reference electrodes. The measurement electrode generates the voltage used to measure a sample solution's pH. The reference electrode includes a barrier configured to screen or separate hydrogen ions from other ions in the solution. The reference and measurement electrodes generate a voltage directly proportional to the pH of the solution. At a pH of 7 (neutral), the electrodes will produce 0 volts between them. At a pH below 7 (acid) the electrodes will produce a voltage of one polarity, and at a pH above 7 (caustic) the electrodes will produce a voltage of the opposite polarity. The magnitude of the voltage will increase in proportion to the difference in logarithmic concentration from a neutral concentration of 10⁻⁷ moles of hydrogen ions per liter. The applications of microelectronic fabrication techniques such as photolithography and thick- and thin-film metallization can be used to produce highly uniform and reproducible pH sensors that are relatively simple to calibrate and operate.

The apparatus can be configured with a combination of sensors that together provide data that can be analyzed to confirm host consumption. For example, the apparatus can be configured with sensors responsive to concentrations of salt, sugars, fats, proteins, light and motion. Movement of the apparatus consistent with chewing can be detected by an accelerometer. For example, a 3-axis accelerometer, such as the LIS3LV02DQ, commercially available from STMicroelectronics, includes a sensing element and an integrated circuit interface that provides acceleration signals. Internal inertial sensors and actuators are implemented in silicon. A concentration of salts, fats, proteins and/or sugars can be determined from a silicon-on-insulator based thin-film resistor. Such a sensor is described in “Silicon-On-Insulator Based Thin-Film Resistor for Chemical and Biological Sensor Applications,” Michael G. Nikolaides, et al., ChemPhysChem 2003, Vol. 4, pgs. 1104-1106, which is incorporated by reference in its entirety. Light can be detected by a charge-coupled device or other photosensors, such as those provided in spectrometers. Data received from an accelerometer can be correlated with one or more measurements of concentrations of salts, fats, proteins and/or sugars in the oral cavity and/or information from one or more photosensors to determine that a host was eating or drinking. Furthermore, collected data can be analyzed to determine the quality and or balance of the dietary constituents in the host's diet.

An intoxilyzer including a broadband infrared source, a narrow-band filter and a photodetector can be included in apparatus 120 to determine when ethanol is present in the host's breath. Infrared (IR) spectroscopy, can be used to identify the presence of specific molecules based on the way they absorb IR light. Molecules are constantly vibrating, and these vibrations change when the molecules absorb IR light. The changes in vibration include the bending and stretching of various bonds. Each type of bond within a molecule absorbs IR at different wavelengths. Thus, to identify ethanol in a sample, you have to look at the wavelengths of the bonds in ethanol (C—O, O—H, C—H, C—C) and measure the absorption of IR light at those specific wavelengths. The absorbed wavelengths help to identify the substance as ethanol, and the amount of IR absorption tells you how much ethanol is present. A conventional formula is then used to determine indirectly the host's blood alcohol content at or near the time when the alcohol was ingested. Alternatively, the length of time associated with the presence of ethanol in the host's breath present in the oral cavity can be used to estimate the volume of alcohol consumed.

Similar measures of the absorption of IR light at other wavelengths can be used to determine the presence of other compounds in the host's oral cavity.

A four-electrode multiple variable sensitive integrated circuit such as that described in “Fish and Chips: Single Chip Silicon MEMS CTDL Salinity, Temperature, Pressure and Light Sensor for Use in Fisheries Research,” A. Hyldgård et al., IEEE MEMS 2005 Proceedings, pg. 303 may be integrated in sensor assembly 430 to generate measurements indicative of conditions in the host's oral cavity.

A photoelectric detector including a light source and a photodetector arranged at an angle (typically 90 degrees) to one another can be used to detect the presence of suspended particles (e.g., smoke) in a host's breath proximal to the apparatus 120. Emitted light is directed such that it normally is not received at the photodetector. When suspended particles are present, the photodetector receives light reflected from the suspended particles.

Outputs from the above-described sensors can be analog current, voltage, or frequency; or digital conversions of the same. In some embodiments, the measurements are recorded in pre-set intervals from a start or reset time commanded via the communication device. In other embodiments, measurements are recorded in intervals that change in accordance with one or more detected conditions in the host's oral cavity or in accordance with the time of day. Recorded information may include an indication of whether a particular substance is present in the oral cavity as determined by comparison of one or more measured conditions with respective thresholds. Alternatively, recorded information may include a measured condition such as a temperature, pH, or concentration of a select substance in the host's oral cavity.

Although measurements are generally, transferred in accordance with an internally generated signal, unscheduled or on-the-spot communication sessions with a suitably configured communication device coupled to a collector can be established to review recently recorded information to determine when the host is under the influence of a recently consumed substance or drug. These real-time tests can be used to determine the host's suitability to perform certain tasks.

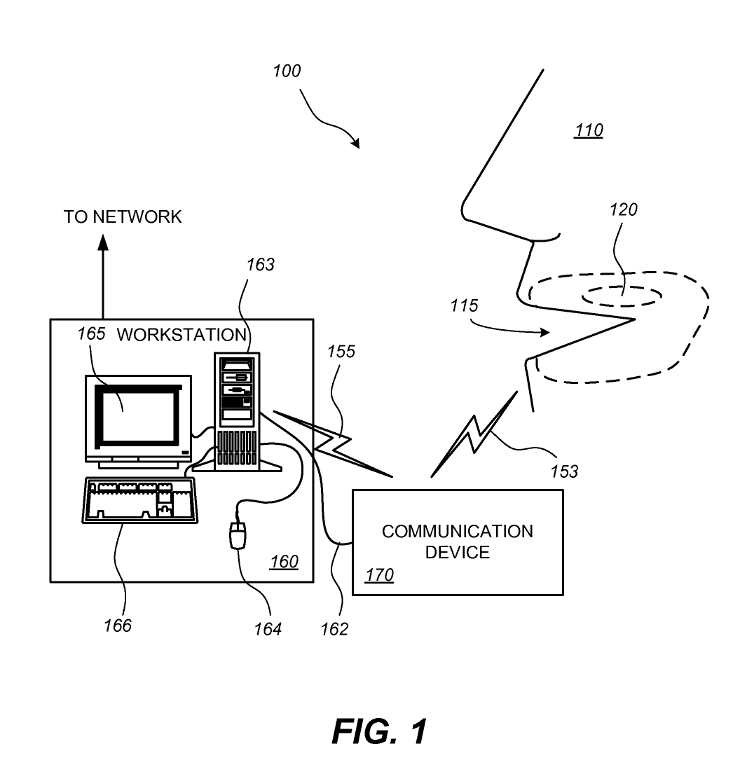

Having generally introduced the apparatuses, systems and methods for monitoring conditions in an oral cavity, various additional embodiments will be described with respect to FIGS. 1-8. By way of example, FIG. 1 is a schematic diagram illustrating components of a system 100 for monitoring conditions in an oral cavity 115. System 100 includes apparatus 120, collector 160 and communication device 170. As described above, apparatus 120 is fixed in the oral cavity 115 of host 110 in a manner that prevents the host 110 from removing apparatus 120.

In the illustrated embodiment, collector 160 is a workstation that includes an enclosure 163 for housing a power supply, central-processing unit, input/output interface controllers as well as fixed and removable data-storage devices. Enclosure 163 is coupled to input/output devices such as mouse 164, display 165 and keyboard 166. Enclosure 163 is coupled to a network via a suitably configured network interface device. The network can be a local-area network, a wide-area network or a combination network. Furthermore, the network interface device can use a wired or a wireless medium to communicate information to network coupled devices.

Collector 160 is further coupled to communication device 170 via wired link 162 or wireless link 155. Communication device 170 is coupled to apparatus 120 via wireless link 153. Wired link 162 can be implemented via any of the packet-based communication protocols commonly known as Ethernet or via one or more standard or proprietary communication protocols for operating a parallel or serial data bus. Wired link 162 may also include circuits for the distribution of power. Wireless link 153 and wireless link 155 can be implemented via any of the short-range radio frequency communication protocols (e.g., 802.11, 802.15.1) or any of the infra-red spectrum based communication protocols (e.g., IrDA).

While wired link 162, wireless link 153 and wireless link 155 are shown together in the illustrated embodiment, one, two or all of the links may be inactive at any given time. For example, most of the time host 110 will not be proximally located to communication device 170. Under these circumstances, a communication session between apparatus 120 and communication device 170 will not be possible because of the limited effective range of the transceivers in the apparatus 120 and communication device 170. When communication device 170 is configured with one or more transceivers that use the infra-red spectrum, communication sessions are possible when host 110 is not only proximally located to communication device 170, but the respective transceivers in apparatus 120 and communication device 170 must be arranged such that the emitter in one is aligned and not obstructed from the infra-red sensitive sensor in the other. When communication device 170 is configured with a self-contained power supply (e.g., a battery) and a memory capacity that exceeds that necessary to store the recorded information in apparatus 120, only one of wireless link 153 or wireless link 155 may be active at any given time.

Collector 160 is configured with software or firmware to transfer and store information received from apparatus 120. Collector 160 is further configured with software suited to organize, display, analyze or otherwise interpret the recorded information.

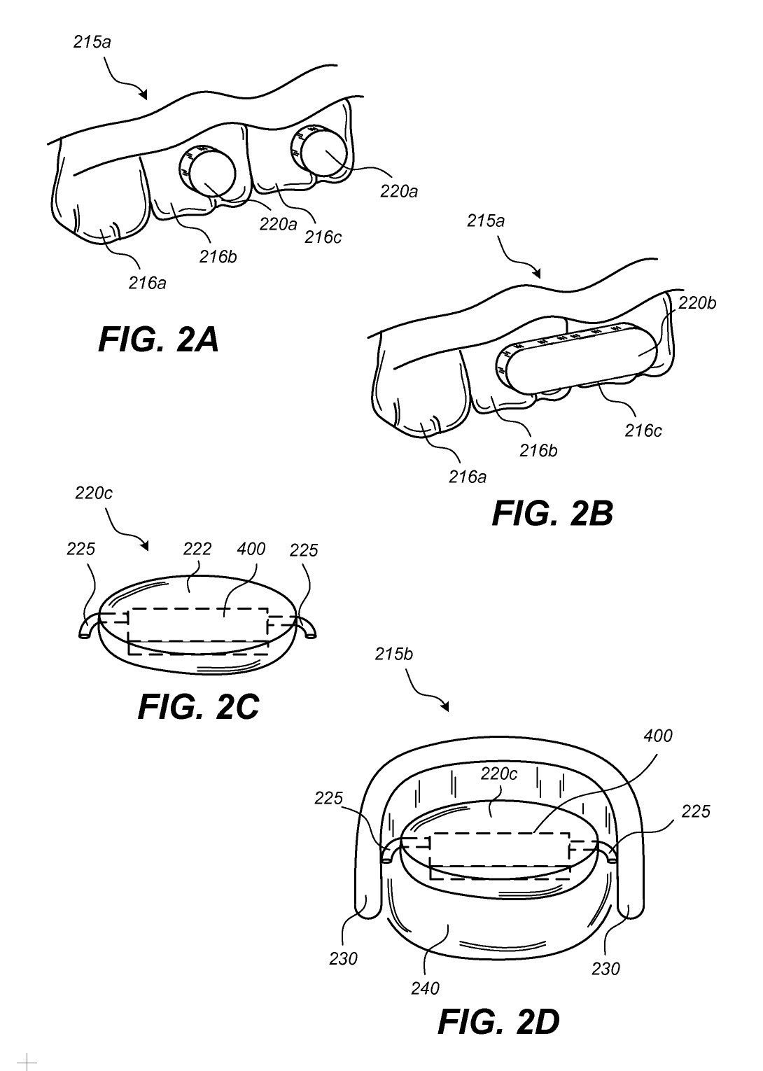

FIGS. 2A through 2D are perspective views of example embodiments of the apparatus of FIG. 1. FIG. 2A is a perspective view of a portion of a host's oral cavity. FIG. 2A illustrates a portion of the upper dentition 215a of host 110. Specifically, the exterior surfaces of molar 216a, molar 216b and molar 216c are shown. Two instances of apparatus 220a are attached to respective molars 216b, 216c. Each instance of apparatus 220a can be bonded to the exterior surface of the corresponding molar using an approved dental adhesive or approved orthodontic fixtures. For example, composite resins, which have become the material of choice for bonding brackets to teeth for orthodontia, can be used to attach the apparatus 220a to the exposed surface. These composite resins can be applied on a mounting surface of the apparatus 220a and positioned on the exterior surface of the molar (e.g., molar 216b) and allowed to cure.

The embodiment of apparatus 220a illustrated in FIG. 2A is generally shaped like a shirt button with the exterior surfaces, other than the mounting surface, being rounded and non-planar. Apparatus 220a is constructed of a material or materials that protect a power supply, an integrated circuit and one or more internal sensors. The exterior surface of apparatus 220a provides a mounting surface for various sensors that detect various select compounds or substances in the host's oral cavity 115 (FIG. 1). Sensors can be positioned along the exterior surfaces of apparatus 220a where they are likely to contact a select or target substance.

Depending on the nature and operation of the sensors, apparatus 220a may be configured with a passageway or channel (not shown) that can receive substances present in the oral cavity 115. The passageway or channel may be protected by a membrane that prevents the physical transfer of bulky foods or other substances that could become stuck or that might otherwise block or interfere with the sensors. Various emitters and detectors associated with the surface positioned sensors may be strategically arranged on opposing surfaces of the channel or on the same surface of the channel depending on the detection mechanism involved (e.g., reflection, absorption, etc. of IR light). The passageway or channel may be arranged with a base surface and opposing side surfaces that extend from the base. In other embodiments, the passageway or channel may be arranged with first and second openings each opposed to the other. In these closed embodiments, one of the two openings has an area that is larger than the other. In this way, food or other substances that encounter apparatus 220a will not become permanently lodged in the apparatus in proximity to the emitters and detectors.

FIG. 2B illustrates the same portion of the upper dentition 215a of host 110 that was presented in FIG. 2A. In FIG. 2B, apparatus 220b is generally capsule shaped and has a length that extends beyond the exterior surface of a single molar. In this embodiment, apparatus 220b is configured with two mounting surfaces (hidden from view in FIG. 2B) that align with an exterior surface of molar 216b and an exterior surface of molar 216c. Apparatus 220b encompasses a larger volume than that encompassed by apparatus 220a. Accordingly, apparatus 220b has a greater capacity for sensors, memory and power supply than that available in apparatus 220a.

In FIG. 2C, apparatus 220c is configured with opposing struts 225 for fixing the apparatus in a host's oral cavity. Struts 225 can be configured with any number of mating fixtures for connecting apparatus 220c in the oral cavity. Struts 225 can be outfitted to interface with brackets, bands or similar devices when the host's oral cavity includes supporting dentition. Alternatively, struts 225 can be outfitted with fixtures configured to interface with posts or other anchors attached to the host's maxilla when the host's oral cavity does not contain sufficient dentition to support the apparatus 220c. Apparatus 220c includes a housing 400 formed in a shell 222 constructed of a dental acrylic or other material approved for use in the oral cavity. Housing 400 encompasses a volume suitable for protecting a power supply, one or more sensors, a controller and memory.

FIG. 2D shows the apparatus 220c of FIG. 2C in arrangement with an abstraction of an upper portion of the host's oral cavity 215c. Specifically, apparatus 220c is positioned in the oral cavity in the space between upper arch 230 and roof 240. It should be understood that FIG. 2D shows the upper portion of the host's oral cavity 215c in an upside down arrangement. When installed in the host's oral cavity, apparatus 220c is actually suspended below roof 240, above the host's tongue (not shown) and between opposing portions of upper arch 230. It should be further understood that struts 225 may be configured in any number of orientations and outfitted with any number of mechanical extensions to permit a semi-permanent installation of apparatus 220c in the host's oral cavity.

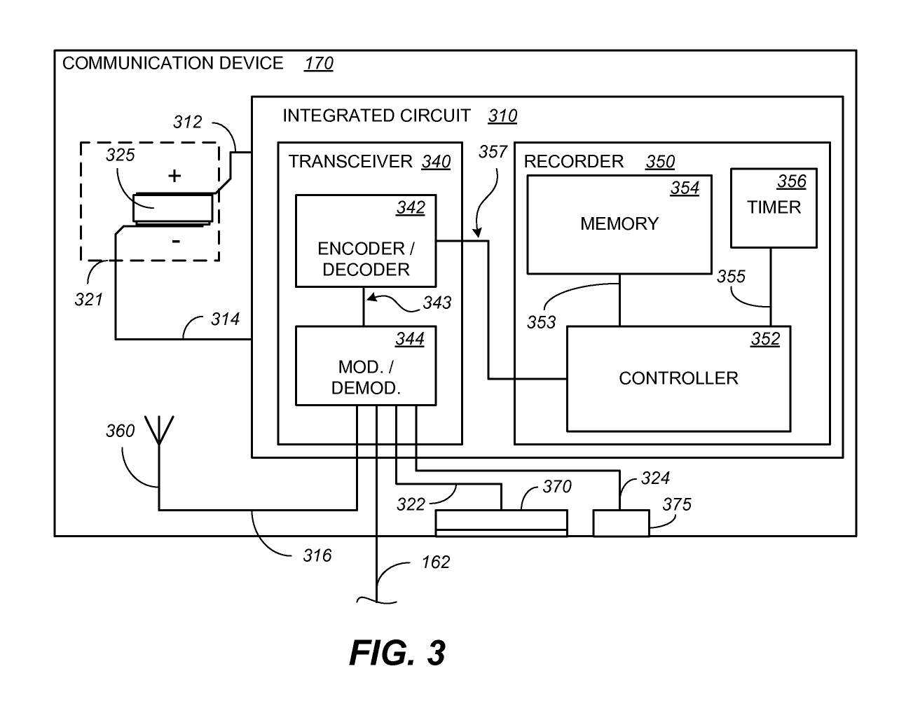

FIG. 3 is a schematic diagram illustrating an embodiment of the communication device 170 of FIG. 1. Communication device 170 includes integrated circuit 310, power source 321, antenna 360, infrared sensor 370 and infrared emitter 380. Power source 321 provides power to integrated circuit 310. Specifically, a positive terminal of power source 321 is electrically connected to a power input pin via conductor 312 and a negative terminal of power source 321 is electrically connected to a ground input pin via conductor 314. In the illustrated embodiment, power source 321 includes battery 325. When communication device 170 is coupled to collector 160 (FIG. 1) conductors within wired link 162 may be dedicated to providing various voltages to integrated circuit 310 in lieu of power supply 321. In addition, wired link 162 may be coupled to a circuit (not shown) configured to charge a rechargeable battery 325 in power supply 321.

Integrated circuit 310 includes recorder 350 and transceiver 340. Recorder 350 is coupled to transceiver 340 via link 357. Transceiver 340 is coupled to antenna 360 via link 316. Transceiver 340 is also coupled to wired link 162 as well as photosensor 370 and light-emitting diode (LED) 375. Photosensor 370 is coupled to transceiver 340 via link 322. LED 375 is coupled to transceiver 340 via link 324. Photosensor 370 detects incident light in the infrared band of frequencies. LED 375 emits light in the infrared band of frequencies in accordance with a modulated signal provided by transceiver 340. LED 375 and photosensor 370 enable line of sight communication via IrDA or other communication protocols that use infrared frequencies to communicate information wirelessly.

Recorder 350 includes controller 352, memory 354 and timer 356. Controller 352 is coupled to memory 354 via bus 353. Controller 352 is further coupled to timer 356 via link 355. Transceiver 340 includes encoder/decoder 342 and modulator/demodulator 344. Encoder/decoder 342 is coupled to modulator/demodulator 344 via bus 343.

In operation, an incident wireless signal containing information is received via antenna 360 and a tuner (not shown) or via photosensor 370. When received via antenna 360, the received signal is forwarded to modulator/demodulator 344 via link 316. When received via photosensor 370, the received signal is forwarded to modulator/demodulator 344 via link 322. When communication device 170 is coupled via a wired connection to a collector 160 (not shown), commands can be forwarded to modulator/demodulator 344 via link 162. Modulator/demodulator 344 detects and separates information from the received signal. The information is forwarded to encoder/decoder 342 via bus 343. Encoder/decoder 342 converts the received information to a format compatible with controller 352. Controller 352 responds in accordance with the one or more received commands. For example, when enabled, communication device 170 is configured to send a request for identifier command. When a suitably configured apparatus 120 is within range and responds with its unique identifier, communication device 170 forwards an command(s) instructing apparatus 120 to communicate recorded information to communication device 170. In turn, communication device 170 will buffer the recorded information until it can be further communicated to collector 160 (not shown). Data transfers between communication device 170 and collector 160 may be interlaced with data being received from apparatus 120.

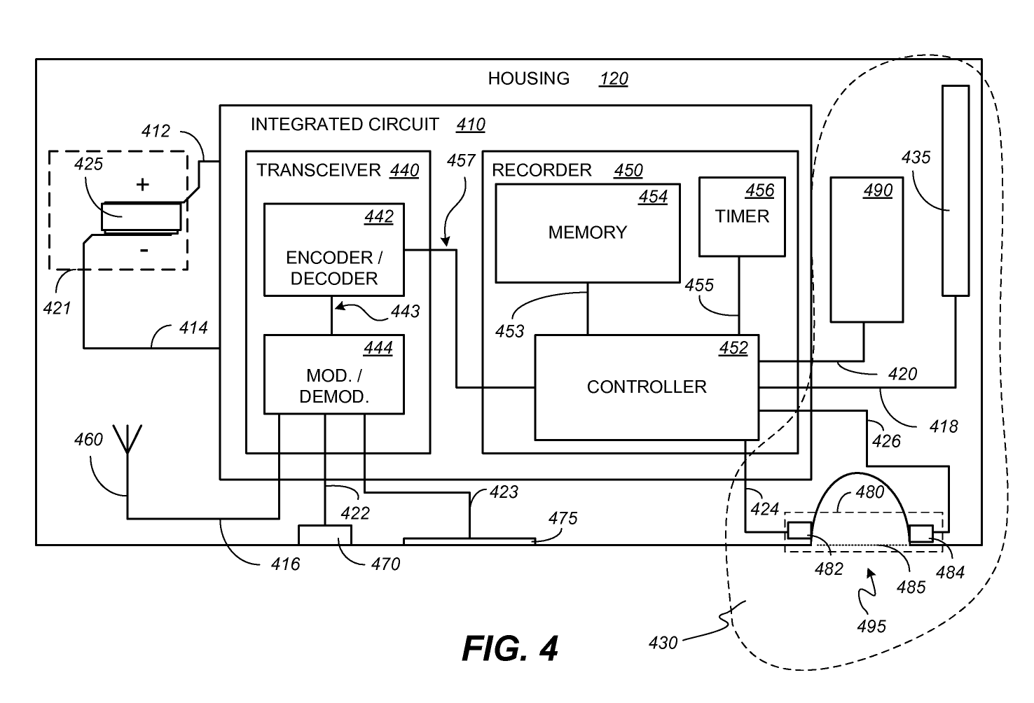

FIG. 4 is a schematic diagram illustrating an embodiment of the apparatus 120 of FIG. 1. Apparatus 120 includes power source 421, sensor assembly 430 and integrated circuit 410. Integrated circuit 410 includes recorder 450 and transceiver 440. In the illustrated embodiment, apparatus 120 entirely encompasses integrated circuit 410, power source 421, and antenna 460. Sensor assembly 430 may include one or more sensors that contact or otherwise interact with the host's oral cavity.

As illustrated in FIG. 4, power source 421 is coupled and provides power to integrated circuit 410. Specifically, a positive terminal of power source 421 is electrically connected to a power input pin via conductor 412 and a negative terminal of power source 421 is electrically connected to a ground input pin via conductor 414.

Integrated circuit 410 includes recorder 450 and transceiver 440. Sensor assembly 430 is coupled to recorder 450 via respective links for each individual sensor component. Sensor 480 is coupled to recorder 450 via link 424 and link 426. Sensor 480 is protected from relatively large substances in the host's oral cavity by membrane 485. Membrane 485 permits the passage of smoke, food or other substances present in the host's oral cavity in portions that are significantly smaller than the width of channel 495. Membrane 485 permits the passage of small amounts of liquid, saliva, and substances partially dissolved therein into channel 495. Sensor 480 includes components along opposing edges of channel 495. Link 424 is coupled to diode 482. Link 424 supplies a signal to energize diode 482. Link 426 is coupled to photosensor 484. Link 426 communicates the received signal from photosensor 484 to controller 452. Sensor 480 is an example of a set of sensors that detect the presence of select molecules in channel 495 using a unique combination of diode 482, excitation signal, and photosensor 484. Other configurations of channel 495 and sensors are possible. Accelerometer 490 is coupled to controller 452 via link 420. Accelerometer 490 communicates information regarding motion in multiple axes to controller 452. Thermometer 435 is coupled to controller 452 via link 418. Thermometer 435 communicates information regarding the temperature of apparatus 120 to controller 452. It should be understood that sensor assembly 430 may contain more or less sensors than thermometer 435, sensor 480, and accelerometer 490.

In the illustrated embodiment, sensor assembly 430 is responsive to temperature, motion and a target or select substance. Target or select substances include salt, fats or lipids, proteins, carbohydrates or sugars including glucose, sucrose, and fructose present in beverages and food in the patient's oral cavity. It should be understood that alternative components and arrangements of sensors within sensor assembly 430 (not shown) may be implemented to detect the presence of dental plaque, drugs, gastrointestinal acid, bacteria, mouthwash, toothpaste, a fluoride rinse, ethanol, bacteria, anti-bodies, among others in the host's oral cavity. Controller 452 can be configured to record measurements from sensor assembly 430 when movement of the oral appliance consistent with chewing is detected and/or when a change of temperature is detected. When one or both of accelerometer 490 and thermometer 435 indicate possible consumption, controller 452 may record measurements from one or more of the remaining sensors in sensor assembly 430 at a reduced interval until such time that consumption is no longer indicated. Once consumption is no longer indicated, controller 452 returns to a default or predetermined interval for recording future conditions in the host's oral cavity. Alternatively, controller 452 can be configured to record measurements in response to timer 456 only.

Recorder 450 includes controller 452, memory 454 and timer 456. Controller 452 is coupled to memory 454 via bus 453. Controller 452 is further coupled to timer 456 via link 455. In operation, signals from timer 456 direct controller to record one or more values from sensor assembly 430. Alternatively, signals from timer 456 direct controller 452 to compare one or more values from sensor assembly 430 with one or more respective thresholds to identify the presence (or lack thereof) of one or more select or target substances.

Transceiver 440 is coupled to recorder 450 via link 457. Transceiver 440 is coupled to antenna 460 via link 416. Transceiver 440 is further coupled to diode 470 via link 422 and infrared sensor 475 via link 423. Transceiver 440 includes encoder/decoder 442 and modulator/demodulator 444. Encoder/decoder 442 is coupled to modulator/demodulator 444 via bus 443.

In operation, a radio-frequency signal or an infrared signal containing one or more commands from a suitably configured communication device is received via antenna 460 or infrared sensor 475 and a tuner (not shown). The received signal is forwarded to modulator/demodulator 444 via link 416. Modulator/demodulator 444 detects and separates information from the received signal. The information is forwarded to encoder/decoder 442 via bus 443. Encoder/decoder 442 converts the received information to a format compatible with controller 452. Controller 452 responds in accordance with the one or more received commands. For example, an identifier set command includes a unique identifier that can be stored in memory 454, a start time reset command includes information responsive to a time or a time and date, an interval set command includes information that defines a time interval between measurements, a transmit command instructs controller 452 to read and communicate each of the recorded measurements, a clear command directs controller 452 to remove recorded measurements from memory 454. A suitably configured communication device 170 may send multiple commands when apparatus 120 is within range of transceiver 440.

During a session, which is defined as the time between a start time reset command and a transmit command, controller 452 in accordance with periodic signals received via link 455 from timer 456 latches a current or a voltage provided by one or more of the sensors in sensor assembly 430.

In some embodiments, controller 452 is configured with an analog to digital converter, which generates a digital representation of the analog output from sensor assembly 430. In these embodiments controller 452 simply forwards the latched and digitized measurement into the next available location within memory 454. In other embodiments, memory 454 is configured with calibration information, which is used to convert the recorded measurement to a scale. When the sensor in these other embodiments is responsive to temperature, the scale may be degrees Fahrenheit or degrees Celsius. In some other embodiments, controller 452 is configured with one or more thresholds that are used to perform a comparison with a measured value in response to a timer output. The result of the comparison, a binary signal or some other symbol is forwarded to the next location in memory 454.

Memory 454 includes adequate storage locations to store measurements for an extended session. When the host 110 of apparatus 120 fails to return within signal range of a suitably configured communication device 170 and controller 452 has forwarded a measurement to each available memory location within memory 454, subsequent measurements will be forwarded to and will overwrite measurement information in the same sequence as previous measurements were stored in memory 454. It should be understood that when apparatus 120 is within range of a communication device measurements can be sent in near real-time from the apparatus 120 to the communication device 170.

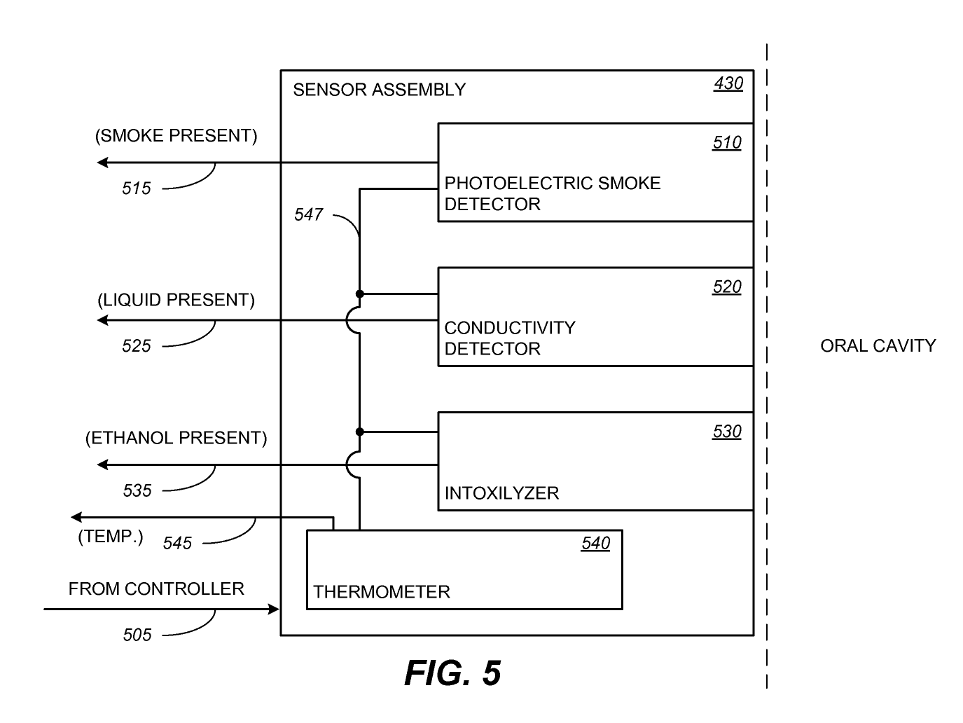

FIG. 5 is a schematic diagram illustrating an embodiment of the sensor assembly 430 of FIG. 4. Sensor assembly 430 receives configuration information and power from controller 452 (FIG. 4) via connection 505. Sensor assembly 430 provides a host of information signals responsive to the presence of one or more substances in the host's oral cavity. Sensor assembly 430 includes various sensors configured to detect target or select substances present in the host's oral cavity and generate corresponding information signals. In the example embodiment, sensor assembly includes thermometer 540, photoelectric smoke detector 510, conductivity detector 520, and intoxilyzer 530.

Thermometer 540 generates a signal responsive to the temperature within the host's oral cavity. Thermometer provided information can be used to determine when the host is smoking, eating or drinking fluids having a temperature that varies from the host's usual temperature in the oral cavity. Thermometer 540 is coupled to photoelectric smoke detector 510, conductivity detector 520, and intoxilyzer 530 via bus 547. Thermometer 540 is also coupled to controller 452 (FIG. 4) via bus 545. The signal from thermometer 540 can be used to enable or otherwise direct substance sensitive sensors such as photoelectric smoke detector 510, conductivity detector 520, intoxilyzer 530 and other sensors (not shown). As further illustrated in FIG. 5, photoelectric smoke detector 510 provides an indication when smoke is present in the host's oral cavity via connection 515. Conductivity detector 520 provides an indication of when a target liquid is present in the oral cavity via connection 525. Intoxilyzer 530 provides an indirect indication of the blood alcohol content of the host by detecting the presence of ethanol molecules in the host's breath in the oral cavity. Intoxilyzer 530 provides the indication via connection 535.

As described above, the indication of the presence or non-presence of a target substance can be communicated as a binary condition after a comparison of a measured value with a predetermined threshold. Alternatively, one or more of the sensors in sensor assembly 430 may forward a measured value for comparison with one or more thresholds stored in controller 452. Although sensor assembly 430 is depicted as a multiple sensor device, it should be understood that for some hosts it may be desired to arrange the sensor assembly 430 with a single sensor.

Regardless of the number of sensors provided in sensor assembly 430, an indication above a threshold value associated with any of the sensors can be used by controller 452 to direct the respective sensor to record information on a more frequent basis than a select interval until a predetermined number of subsequent measurements indicate that the select substance is no longer present in the oral cavity. In this way, apparatus 120 can provide information with greater resolution than that provided with a single measurement period. In addition, the power supply can be conserved and the memory capacity used more efficiently than with a single measurement period.

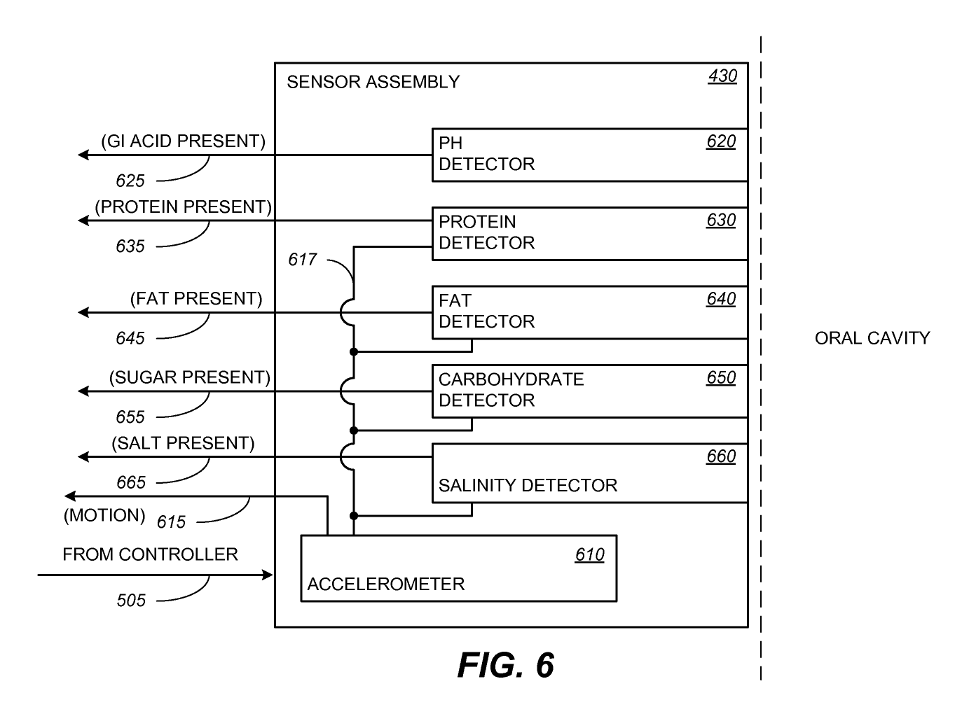

FIG. 6 is a schematic diagram illustrating an alternative embodiment of the sensor assembly 430 of FIG. 4. Sensor assembly 430 receives configuration information and power from controller 452 (FIG. 4) via connection 505. Sensor assembly 430 provides a host of information signals responsive to the presence of one or more substances in the host's oral cavity. Sensor assembly 430 includes various sensors configured to detect target or select substances present in the host's oral cavity and generate corresponding information signals. In the example embodiment, sensor assembly includes accelerometer 610, pH detector 620, protein detector 630, fat or lipid detector 640, carbohydrate (sugar) detector 650, and salinity detector 660.

Accelerometer 610 generates signals responsive to the rate of motion of housing 400 (FIG. 4). Accelerometer 610 provided information can be used to determine when the host is eating, sleeping, or awake but not eating. Accelerometer 610 is coupled to protein detector 630, fat detector 640, carbohydrate detector 650 and salinity detector 660 via bus 617. Accelerometer 610 is also coupled to controller 452 (FIG. 4) via bus 615. Signals from accelerometer 610 can be used to enable or otherwise direct food substance sensitive sensors such as protein detector 630, fat detector 640, carbohydrate detector 650 and salinity detector 660. As further illustrated in FIG. 6, pH detector 620 provides an indication of when gastrointestinal acid is present in the oral cavity via connection 625. Protein detector 630 provides an indication of when a target protein is present in the oral cavity via connection 635. Fat detector 640 provides an indication when a target fat is present in the oral cavity via connection 645. Carbohydrate detector 650 provides an indication when a target sugar is present in the oral cavity via connection 655. Salinity detector 660 provides an indication when salt is present in the oral cavity via connection 665.

As described above, the indication of the presence or non-presence of a target substance can be communicated as a binary condition after a comparison of a measured value with a predetermined threshold. Alternatively, one or more of the sensors in sensor assembly 430 may forward a measured value for comparison with one or more thresholds stored in controller 452. Sensor assembly 430 can be configured with other combinations of sensors sensitive to compounds other than those shown in FIGS. 5 and 6. For example, sensor assembly 430 can be configured with a sensor that indicates the presence of therapeutic medications as well as the presence of illicit drugs either directly present in the oral cavity or indirectly via an exhaled byproduct in the host's breath. Moreover, sensor assembly 430 can be configured with a sensor that indicates the presence of unhealthy bacteria, a sign of non-compliance with practices consistent with good oral hygiene.

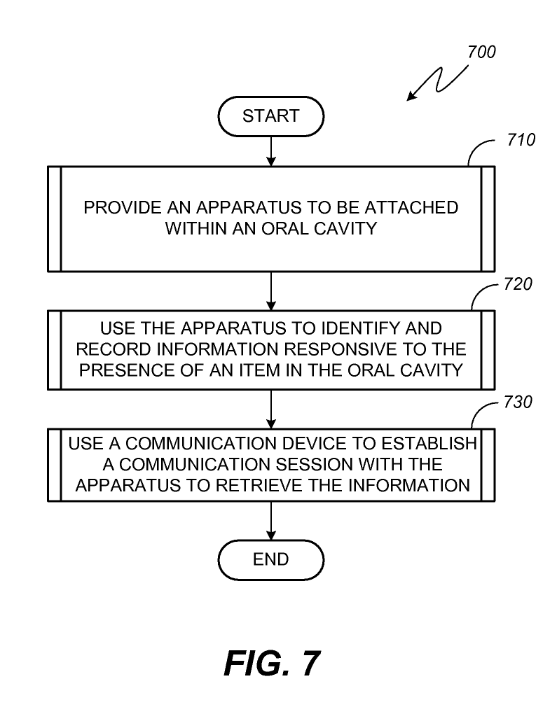

FIG. 7 is a flow diagram illustrating an embodiment of a method 700 for monitoring conditions in an oral cavity. In this regard, the functions associated with block 730 represent respective specified steps or functions that can be embodied in software and/or a combination of hardware and firmware. When embodied in software and/or hardware/firmware, the functionality described in block 730 can be embodied in modules, segments, or portions of code, which comprise one or more executable instructions for implementing the specified function(s).

Method 700 begins with block 710 where an apparatus configured for attachment within an oral cavity is provided to a party interested in monitoring the consumption habits of a host over time. Installation of the device may be associated with a reset procedure or other initialization of the apparatus. This may be as simple as installing a battery and/or establishing a communication session with the apparatus proximal to the time when a healthcare professional installs the apparatus in the host's oral cavity to set an initial time and transfer various operating parameters to the apparatus. As described above, the provided apparatus is configured to record a measurement or measurements indicative of the presence of one or more target substances or compounds in the host's oral cavity. Upon installation in the host's oral cavity and as indicated in block 720, the apparatus is used to identify and record information responsive to the presence of a substance in the oral cavity.

As described above, the recorded information will include an indication of the time when the measurement was recorded. The information may be provided in the form of a binary indicator responsive to a comparison between a predetermined threshold stored in the apparatus and a corresponding output of a sensor configured to identify a characteristic associated with a select or target substance. The recorded information can also be provided in the form of a measured value associated with a characteristic of the target substance. When the recorded information is a measured value, additional recorded values may be reviewed or otherwise analyzed by collector 160 (FIG. 1) or other devices communicatively coupled to collector 160. In preferred embodiments, the apparatus is configured to periodically record one or more specified measurements or one or more present/not present conditions over a select length of time that does not exceed the capacity of a memory device to save the information. Once the host of the apparatus is within range of a suitably configured communication device, as indicated in block 730, the communication device is used to establish a communication session with the apparatus to retrieve the recorded information.

One or more operational software programs that may be used by a communication device, as well as operational software programs that may be used in conjunction with a computing device communicatively coupled to the communication device, which comprise an ordered listing of executable instructions for implementing logical functions, can be embodied in any computer-readable medium for use by or in connection with an instruction execution system, or device, such as a computer-based system, processor-containing system, or other system that can fetch the instructions from the instruction execution system, apparatus, or device and execute the instructions. Consequently, portions of the described method and alternatives can be embodied on a computer-readable medium.

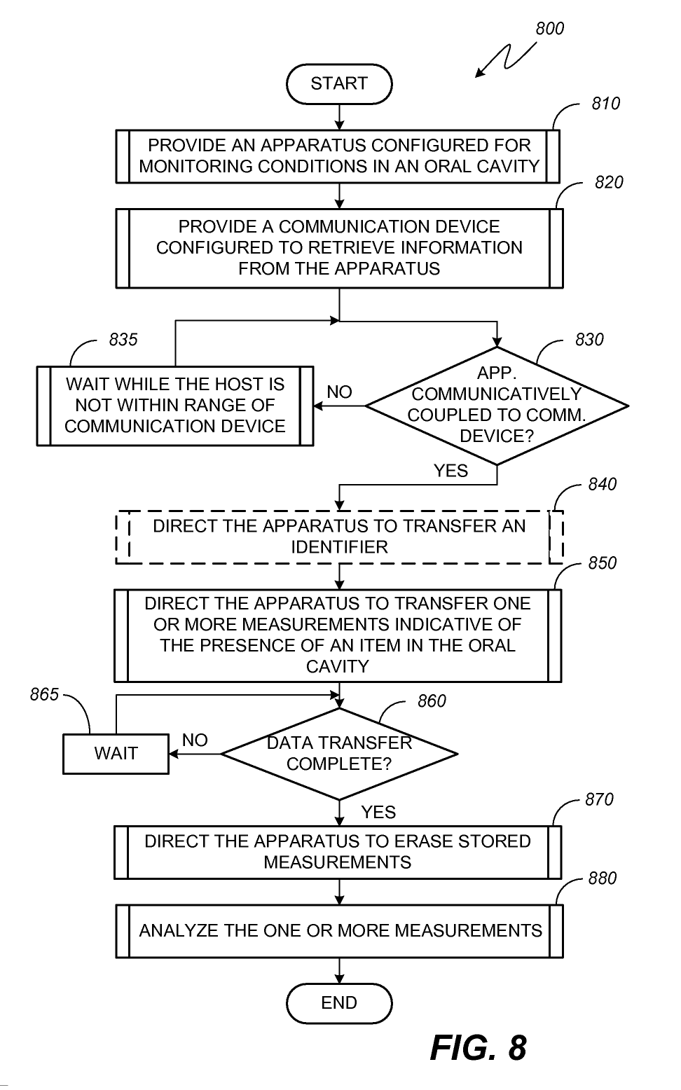

FIG. 8 is a flow diagram illustrating an alternative embodiment of a method 800 for monitoring conditions in an oral cavity. In this regard, each block represents a specified step or function. When embodied in software and/or hardware/firmware, each block represents a module, segment, or portion of code, which comprises one or more executable instructions for implementing the specified function(s).

Alternative method 800 begins with block 810 where an apparatus is provided that can be fixed or installed in a host's oral cavity. The apparatus is configured to monitor conditions in the oral cavity to identify the presence or lack of one or more specified substances or compounds.

In block 820, a communication device is provided that can retrieve information recorded in the apparatus. As described above, the apparatus periodically records one or more indications of the presence or lack thereof of a target or select substance. The apparatus continues to record information as long the power supply contained therein is not exhausted. When the capacity of the internal memory is exceeded, the apparatus is configured to overwrite the earliest recorded information stored in the internal memory. As indicated in decision block 830 a determination is made whether the apparatus is communicatively coupled to a suitably configured communication device. When the apparatus is not communicatively coupled to the communication device as indicated by the flow control arrow labeled “NO” exiting decision block 830 processing continues with block 835 where one waits while the host is not within range or is otherwise not coupled to the communication device.

Otherwise, when the apparatus is communicatively coupled to the communication device, as indicated by the flow control arrow labeled “YES” exiting decision block 830 processing continues with one or both of blocks 840 and 850. In block 840, the communication device directs the apparatus to transfer an identifier that was previously assigned to the apparatus and associated with one or more identifiers associated with the host in which the apparatus is installed. In block 850, the communication device directs the apparatus to transfer one or more measurements indicative of the presence of a substance (or the lack thereof) in the host's oral cavity. Preferably, the communication device directs the apparatus to transfer the entire set of recorded information from the time when the apparatus was first installed or since the last communication session during which data was transferred from the apparatus. As indicated in decision block 860, block 865 and the adjacent flow control arrows, the communication device and the apparatus continue to communication session until the communication link is no longer available or all data has been transferred. Thereafter, as indicated in block 870 the communication device directs the apparatus to erase stored measurements. In alternative embodiments, communication device 870 may simply reset a write counter and the timer to directs a memory controller to store subsequent measurements at a desired location in the apparatus memory. Once the recorded information has been transferred, the recorded information can be analyzed as indicated in block 880. As described above, analysis of the communicated data may include comparison with one or more prescribed therapy schedules.

While various embodiments of the apparatuses, systems and methods for monitoring conditions in an oral cavity have been described, it will be apparent to those of ordinary skill in the art that many more embodiments and implementations are possible that are within the scope of the accompanying claims. Accordingly, the apparatuses, systems and methods for monitoring conditions in an oral cavity are not to be restricted beyond the attached claims and their equivalents.

What is claimed is:

1. An apparatus for monitoring an oral cavity, comprising:
a housing configured to be mounted within an oral cavity, the housing comprising:
a power source;
a sensor assembly powered by the power source;
a memory coupled to the sensor assembly and configured to store information responsive to the presence of a substance in the oral cavity; and
a transceiver coupled to the memory and configured to communicate the information in accordance with a received command.

2. The apparatus of claim 1, wherein the sensor assembly generates a first signal responsive to the presence of a cloud of fine particles suspended in a gas proximate the sensor assembly.

3. The apparatus of claim 1, wherein the sensor assembly generates a second signal responsive to movement consistent with consumption.

4. The apparatus of claim 3, wherein the sensor assembly generates a third signal responsive to a target constituent of a food.

5. The apparatus of claim 3, wherein the sensor assembly generates a fourth signal responsive to a target liquid.

6. The apparatus of claim 3, wherein the sensor assembly generates a fifth signal responsive to a threshold concentration of a carbohydrate.

7. The apparatus of claim 3, wherein the sensor assembly generates a sixth signal responsive to a threshold concentration of sodium chloride.

8. The apparatus of claim 3, wherein the sensor assembly generates a seventh signal responsive to a threshold concentration of a lipid.

9. The apparatus of claim 3, wherein the sensor assembly generates an eighth signal responsive to a threshold concentration of a protein.

10. The apparatus of claim 1, wherein the sensor assembly comprises an intoxilyzer.

11. The apparatus of claim 1, wherein the sensor assembly is sensitive to a drug.

12. The apparatus of claim 1, wherein the sensor assembly is sensitive to a threshold concentration of gastrointestinal acid.

13. A system for monitoring conditions in an oral cavity, comprising:
an apparatus attached within the oral cavity, the apparatus configured to identify and store information responsive to the presence of a substance in the oral cavity;
a communication device configured to establish a communication session with the apparatus and direct the apparatus to communicate the information; and
a collector configured to receive the information from the communication device.

14. The system of claim 13, wherein the communication device establishes the communication session via a wireless medium.

15. The system of claim 13, further comprising an interface configured to associate the information with time and present a composite representation of the information over a desired period of time.

16. The system of claim 13, wherein the apparatus is sensitive to the presence of a byproduct.

17. The system of claim 13, wherein the apparatus is sensitive to a characteristic of a consumed substance.

18. A method for monitoring conditions in an oral cavity, comprising:
providing an apparatus suitable for attachment within an oral cavity;
using the apparatus to identify and record information responsive to the presence of a substance in the oral cavity;
using a communication device to establish a communication session with the apparatus to retrieve the information.

19. The method of claim 18, further comprising:
using the retrieved information to confirm that a host of the apparatus is complying with a diet.

20. The method of claim 18, further comprising:
using the retrieved information to confirm that a host of the apparatus is not smoking.

21. The method of claim 18, further comprising:
using the retrieved information to confirm that a host of the apparatus is complying with a schedule for oral hygiene.

22. The method of claim 18, further comprising:
using the retrieved information to confirm that a host of the apparatus is not presently under the influence of an orally consumed drug.

23. The method of claim 18, further comprising:
using the retrieved information to confirm that a host of the apparatus is not consuming a target substance over a select period of time.

24. The method of claim 18, further comprising:
using the retrieved information to confirm that a host of the apparatus is suffering from an eating disorder.

25. The method of claim 18, further comprising:
using the retrieved information to confirm that a host of the apparatus is suffering from gastro-esophageal reflux disease (GERD).
